(19) United States
(12) Patent Application Publication
(10) Pub. No.: US 2016/0058600 A1
(43) Pub. Date: Mar. 3, 2016

(54) DAMAGE-REDUCING CERVICAL COLLAR

(71) Applicant: John William Basill, St. Louis Park, MN (US)

(72) Inventor: John William Basill, St. Louis Park, MN (US)

(21) Appl. No.: 14/474,159

(22) Filed: Aug. 31, 2014

(51) Int. Cl. A61F 5/055

(52) U.S. Cl. CPC A61F 5/055

(57) ABSTRACT

A neck support collar may have:
a resilient collar base with a lower edge and a top edge and two minor sides between the lower edge and the top edge;
the top edge being relatively more elevated in its middle between the two minor sides;
at least three resilient ribs of at least 15 centimeters extending from the lower edge of the of the collar base;
each resilient rib being elastic and having a yield point of at least 50 kilograms at a 90 degree deflection on a 15 cm rib; and
the ribs extending at least 5 cm below the lower edge and at least 5 cm into the resilient collar base.

### BACKGROUND OF THE INVENTION

1. Field of the Invention

The present invention relates to the field of neck collars that are worn by people, especially during sports activities, where the collar is designed to reduce whiplash soft tissue damage, especially low-to-moderate-impact events.

2. Background of the Art

Cervical whiplash syndrome, or hyperextension-hyperflexion injury, is a common traumatic injury in sports activities. The mechanisms of injury that cause cervical whiplash syndrome vary, but they may be sufficient enough to cause chronic whiplash syndrome.

More than 3.5 million teenagers and children, ages 14 and under, are injured every year by playing competitive and individual sports, as well as by participating in simple athletic recreational activities.

Sports and recreational athletic activities are responsible for more than 21 percent of all traumatic brain injuries among teenagers, children and adolescents annually in the United States.

More than 60 percent of head injuries that occur during athletic competitions or recreational activities happen during bicycling, skateboarding, or skating related sports.

More than 800,000 teenagers and children, ages 14 and under, are admitted to emergency rooms each year because of injuries related to athletic competition and recreational activities. The majority of these injuries happen because of a striking object, collisions, falling, or physical overextertion.

Of non-fatal injuries that are treated in hospital emergency rooms each year, the majority are caused by athletic competition or recreational activities.

Many different devices have been designed to address these injuries as either protective devices or as prophylaxis devices attempting to prevent injuries. These devices range from simple foam collars, to foam collars with design features (as shown in U.S. Pat. No. 8,267,877 [Sandhu]; U.S. Pat. No. 6,874,170 [Aaron]; U.S. Pat. No. 6,862,749 [Krause]; U.S. Pat. No. 6,058,517 [Hartunian]; U.S. Pat. No. 5,275,581 [Bender, showing internal vertical support splints]; U.S. Pat. No. 3,964,474 [Fox]; U.S. Pat. No. 3,320,950 [McElvenny]; U.S. Pat. No. 3,189,917 [Sims]; U.S. Pat. No. 2,806,471 [Breese]; and U.S. Pat. No. 2,223,276 [Ward]) up to more complex systems having shoulder pads, chest harnesses and helmets (as shown in U.S. Pat. No. 8,740,830 [Suarez]; U.S. Pat. No. 8,715,212 [Ely]; U.S. Pat. No. 8,615,819 [Kerr]; U.S. Pat. No. 5,141,489 [Sereboff]; U.S. Pat. No. 4,996,720 [Fair]; U.S. Pat. No. 4,501,023 [Bilberry] and U.S. Pat. No. 2,820,455.

These prior art systems are either very simple and offer little more than cosmetic effects where essentially foam-only collars are used, or are complex, expensive and movement restrictive. It is desirable to be able to provide a simple device that can provide a defined degree of possible protection against at least low-impact whiplash events without overly restricting player movements. All documents cited herein are incorporated bt reference in their entirety.

### SUMMARY OF THE INVENTION

A neck support collar may have: a resilient collar base with a lower edge and a top edge and two minor sides between the lower edge and the top edge; the top edge being relatively more elevated in its middle between the two minor sides; at least three resilient ribs of at least 15 centimeters extending from the lower edge of the of the collar base; each resilient rib being elastic and having a yield point of at least 50 kilograms at a 90 degree deflection on a 15 cm rib; and the ribs extending at least 5 cm below the lower edge and at least 5 cm into the resilient collar base.

### BRIEF DESCRIPTION OF THE FIGURES

FIG. 1 shows a perspective view of a neck support collar according to the present technology.

FIG. 2 shows a front view of a neck support collar according to the present technology.

FIG. 3 shows a side view of a neck support collar according to the present technology.

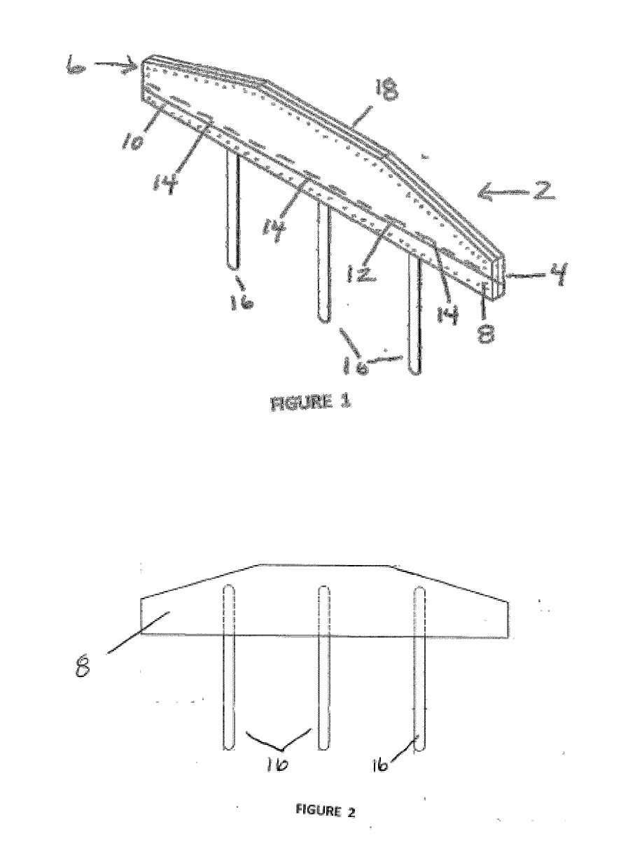

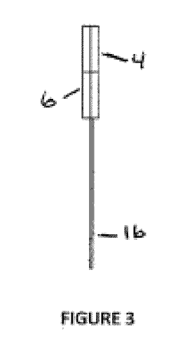

### DETAILED DESCRIPTION OF THE INVENTION

A neck support collar may have: a resilient collar base with a lower edge and a top edge and two minor sides between the lower edge and the top edge; the top edge being relatively more elevated in its middle between the two minor sides; at least three resilient ribs of at least 15 centimeters extending from the lower edge of the of the collar base; each resilient rib being elastic and having a yield point of at least 50 kilograms at a 90 degree deflection on a 15 cm rib; and the ribs extending at least 5 cm below the lower edge and at least 5 cm into the resilient collar base.

The resilient collar base is preferably an elastic polymeric material with substantive resilient strength that can provide flexible support to the neck and head of a human. That resilience is sufficient to resist or limit some reasonable degree of sudden backwards head movement, while allowing the wearer to move the head to a controlled degree. Leaf spring elements such as stiff and flexible stays can provide such performance. Leaf springs display this type of performance characteristic, increasing resistance to movement as the leaf spring is flexed to a greater degree.

Some particular polymer compositions include fiber-reinforced polymers, rib reinforced polymer sheeting (see Published US Patent Application Document No. 20130196120, Gray) or a closed cell memory foam having a density of at least 4.0 lbs/ft³. The resilient collar base may have a compression set of less than 5% at 90% compression, for 22 hours at 158° F., or a compression set of less than 1% at 90% compression, for 22 hours at 158° F. The resilient collar base may be made of a closed cell memory foam having a density of at least 4.0, 4.5, 5.0, 5.5 or at least 6.0 lbs/ft³. The collar base may have a replaceable sleeve that covers the resilient collar base and has openings that allow the at least three resilient ribs to extend out of the sleeve by at least 5 cm. The sleeve should be washable, as with a nylon or polyester fabric cover. The collar may have the at least three resilient ribs with at least 10, at least 15, at least 18, at least 20, at least 25 or at least 30 centimeters in length with at least 12 cm, at least 15 cm or at least 20 cm extending from the lower edge of the of the collar base, with or without fabric covering. At least one rib must remain between the shoulder blades when the collar is attached. Preferably at least two and more preferably at least three ribs should remain between the shoulder blades at the back of the wearer when the collar is secured.

The ribs may also be adjustable and replaceable within the collar base. The ribs extend into the collar base and may be permanently attached or slid into slots or cavities fit for the ribs. The ribs can be slid and even locked into position (by tightening screws, or a sequence of laddered horizontal ribs that progressively snap into place to adjustably lock the rib at a depth in the cavity. In this way, both comfort and resilient strength (shorter ribs offer greater resistance to flexing, while longer ribs provide less resistance but longer support area against the wearer's back). The collar preferably has a replaceable sleeve that covers the resilient collar base and has openings therein that allow the at least three resilient ribs to extend out of the sleeve by at least 10, at least 15 or at least 20 cm.

The collar may be formed with each resilient rib being elastic and having a yield point of at least 50, at least 60, at least 70 or at least 80 kilograms at a 90 degree deflection on a 15 cm rib secured at one end and the force applied at the other end continually perpendicular at the 15 cm distance. The collar sleeve may have an opening along a face of the collar between the top edge and the lower edge and a hook and loop fastener temporarily secures sleeve material along the opening. The collar base I slid into the sleeve, and the ribs pass through holes in the bottom of the sleeve, or the ribs may be inserted through the holes into the collar base through the holes in the sleeve. The sleeve may have an opening along a face of the collar between the top edge and the lower edge and a hook and loop fastener temporarily secures sleeve material along the opening.

The elastic limit of a material is an important consideration in civil, mechanical, and aerospace engineering and design. Elastic limit, also referred to as yield point, is an upper limit for the stress that can be applied to a material before it permanently deforms. This limit is measured in pounds per square inch (psi) or Newtons per square meter, also known as pascals (Pa).

Elastic limit is a function of the elasticity of a material. Elasticity is the ability of a material to return to its original shape, or dimensions, after a load or stress is removed. All materials will deform when a stress or load is applied. Strain is a measure of the amount of deformation that occurs when a material is under stress.

Elastic strain occurs when a material is exposed to low stress. It will disappear after the stress is removed, and the material will return to its original state. Plastic strain will occur at stresses above the elastic limit. A material that experiences plastic strain will not fully recover and return to its original dimensions after the stress is removed.

High density foam is a type of polymeric foam (e.g., polyurethane, polyolefin, polysilixoane, etc.) that is composed of a large amount of open cells or bubbles that are packed tightly together. The result of this higher concentration or density is that air is able to pass through the holes in the cells with greater ease, making it much easier to provide comfortable padding for cushions, mattresses, and other similar products. Foam density of this type also tends to hold its shape with greater efficiency, allowing it to return to normal dimensions when any object is lifted from the surface of the foam.

One of the best ways to understand how high density foam works is to consider a mattress that is manufactured using the product. The structural foam used in the mattress allows it to conform to the contours of the individual sleeping on the mattress. At the same time, the foam provides support that allows the spine to remain in a natural position, eliminating stress on the back and neck. As a result of this cushioning effect, the individual is able to enjoy a deeper and more recuperative sleep, and is more likely to awake feeling rested and refreshed.

Unlike some other products, high density foam has the ability to regain the original shape shortly after any weight is lifted from the product. For example, a mattress made with this foam provides ideal support for the sleeper, but quickly reverts to its original appearance after the individual arises. Such mattresses will hold their shape for many years, providing a longer period of active use than mattresses using other types of materials.

There are several other common uses of high density foam. Cushions and pillows are often made with this product. Upholstery foam that is high density allows sofas and chairs to provide comfortable sitting for many years before any sagging or indentations begin to appear underneath the upholstery. The product can also be used for wall padding in recording booths and other settings, helping to muffle noises with great efficiency.

In terms of cost, products made with high density foam tend to be competitive with products manufactured using other materials. Often, the foam products can equal or even exceed the life of products made with other materials. For this reason, using this product for padding or as the main material in a number of products is both practical and cost-effective for manufacturers, as well as being affordable and offering long-term service to consumers.

Foam densities (lb/ft³), resilience (%), compression set (% at 90% compression, for 22 Hours at 158° F.) and tensile strength (lb/in2) are useful characterization factors for the properties of foam.

Low density (less than 2.0 lb per cubic feet) foams are highly resilient, light weight and offer minimal soft cushioning effects. Intermediate density (greater than 2.0 and less than 3.5 lbs per cubic feet) has more strength and offers greater resistance to compression, and is often found in mattresses and cushions for human use. Higher (at least 4.0 lb/ft³) and high density foam (between at least 5.0 and up to or exceeding 7.5 lb/ft³) has capability of more intense structural use in medical support devices and even paneling for furniture.

The present cervical collar base preferably uses higher density memory foam (often referred to as medical foam) having a density of at least 4.0 lb/ft³, at least 5.0 lb/ft³, or at least 6.0 lb/ft³, or at least 7.0 lb/ft³. The foam should have a compression set of less than 5% at 90% compression, for 22 hours at 158° F. The foam preferably has a compression set of less than 3%, less than 2%, less than 1% and less than 0.5% under those conditions. The foam preferably has a tensile strength of at least 10 lbs/in², at least 15 10 lbs/in², and at least 20 lbs/in².

Although specific materials, dimensions and properties have been described, alternatives and equivalents may be practiced by those skilled in the art and remain in the scope of the present invention.

What is claimed:

1. A neck support collar comprising:
a resilient collar base having a lower edge and a top edge and two minor sides between the lower edge and the top edge;
the top edge being relatively more elevated in its middle between the two minor sides;
at least three resilient ribs of at least 15 centimeters extending from the lower edge of the of the collar base;
each resilient rib being elastic and having a yield point of at least 50 kilograms at a 90 degree deflection on a 15 cm rib;
the ribs extending at least 5 cm below the lower edge and at least 5 cm into the resilient collar base.

2. The collar of claim 1 wherein the resilient collar base is a closed cell memory foam having a density of at least 4.0 lbs/ft³.

3. The collar of claim 2 wherein the resilient collar base has a compression set of less than 5% at 90% compression, for 22 hours at 158° F.

4. The collar of claim 2 wherein the resilient collar base has a compression set of less than 1% at 90% compression, for 22 hours at 158° F.

5. The collar of claim 4 wherein the resilient collar base is a closed cell memory foam having a density of at least 6.0 lbs/ft³.

6. The collar of claim 1 having a replaceable sleeve that covers the resilient collar base and has openings that allow the at least three resilient ribs to extend out of the sleeve by at least 5 cm.

7. The collar of claim 6 wherein the at least three resilient ribs are at least 20 centimeters in length with at least 12 cm extending from the lower edge of the of the collar base;

8. The collar of claim 4 having a replaceable sleeve that covers the resilient collar base and has openings that allow the at least three resilient ribs to extend out of the sleeve by at least 5 cm.

9. The collar of claim 8 wherein the resilient collar base is a closed cell memory foam having a density of at least 6.0 lbs/ft³.

10. The collar of claim 2 wherein each resilient rib being elastic and having a yield point of at least 75 kilograms at a 90 degree deflection on a 15 cm rib.

11. The collar of claim 4 wherein each resilient rib being elastic and having a yield point of at least 75 kilograms at a 90 degree deflection on a 15 cm rib.

12. The collar of claim 5 wherein each resilient rib being elastic and having a yield point of at least 75 kilograms at a 90 degree deflection on a 15 cm rib.

13. The collar of claim 6 wherein each resilient rib being elastic and having a yield point of at least 75 kilograms at a 90 degree deflection on a 15 cm rib.

14. The collar of claim 8 wherein each resilient rib being elastic and having a yield point of at least 75 kilograms at a 90 degree deflection on a 15 cm rib.

15. The collar of claim 6 wherein the sleeve has an opening along a face of the collar between the top edge and the lower edge and a hook and loop fastener temporarily secures sleeve material along the opening.

16. The collar of claim 7 wherein the sleeve has an opening along a face of the collar between the top edge and the lower edge and a hook and loop fastener temporarily secures sleeve material along the opening.

17. The collar of claim 8 wherein the sleeve has an opening along a face of the collar between the top edge and the lower edge and a hook and loop fastener temporarily secures sleeve material along the opening.
